(19) United States
(12) Patent Application Publication
(10) Pub. No.: US 2026/0091076 A1
(43) Pub. Date: Apr. 2, 2026

(54) FORMULATION AND METHOD FOR MANAGING AND TREATING INFLAMMATORY MACROPHAGE-INVOLVED BOWEL DISEASES

(71) Applicant: Physis International LLC, Westerville, OH (US)

(72) Inventors: Frederick O. Cope, Farragut, TN (US); Michael S. Blue, Galena, OH (US); Simon A. Blackburn, New Albany, OH (US)

(21) Appl. No.: 19/344,426

(22) Filed: Sep. 29, 2025

Related U.S. Application Data
(60) Provisional application No. 63/700,143, filed on Sep. 27, 2024.

(51) Int. Cl. A61K 38/01; A61K 9/00; A61K 35/60; A61K 47/12; A61K 47/24; A61K 47/26

(52) U.S. Cl. CPC A61K 38/011; A61K 9/0053; A61K 35/60; A61K 47/12; A61K 47/24; A61K 47/26

(57) ABSTRACT

Formulation and method for convalescing the status of the gastrointestinal tract in vertebrate subjects expressing a symptomology characteristic of inflammatory bowel diseases. The formulation and method based on the effective targeting of lectin receptors expressed by disease-contributing inflammatory macrophages that are known pathological elements of inflammatory bowel diseases' symptomology in vertebrate animals.

### CROSS-REFERENCE TO RELATED APPLICATIONS

This application claims the benefit of U.S. Provisional Application No. 63/700,143 filed Sep. 27, 2024, the disclosures of which are hereby incorporated by reference as if fully restated herein.

### FIELD

Exemplary embodiments relate generally to compositions and methods for treating and managing bowel diseases involving macrophage cells, particularly those expressing some element of the inflammatory phenotypes.

### BACKGROUND

Inflammatory bowel disease (IBD), comprised primarily of ulcerative colitis and Crohn's disease, is a chronic relapsing cyclic inflammatory disease of the gastrointestinal tract (GI) that is related to immune modulation. Macrophages are the key agent of GI immune homeostasis and function, and have existential influence on disease natural history, particularly the inflammation process, the microbiome, cytokine production, chemokine expression, and non-immune cell gene expression and function. This impact leads to profound clinically pathological GI conditions. Hence, macrophages have been recognized as attractive targets to develop new therapeutic approaches for the disease.

Metabolism reprogramming is a key determinant that dictates macrophage functions and subsequent disease progression. This approach specifically deals with phenotypic alteration of the macrophage population such that GI macrophage populations alter their gene expression profiles characteristic of their well-known non-inflammatory states.

Many approaches to modulating the macroenvironments and microenvironments of the GI tract in order to mitigate (treat) and manage the IBD have focused on the microbiome, cytokine production, or other cell product as the functional culprits of IBD. However, expression of such constituents may not effectively treat IBD.

Metabolic alterations in the form of cytokines and other cell products are constituent to elements of IBD.

Numerous inventions have attempted to approach the clinical symptomology of inflammatory bowel diseases by focusing on altering the microbiome of the gastrointestinal tract. Such an approach presumes that the leading elements of the inflammatory bowel disease conditions lay within the bacterial population distribution of the gastrointestinal tract. However, none of these approaches addresses the root elements of the involvement of the immune system and the specificity of the pathological characters, namely inflammatory macrophages and their intermediate phenotypes involved in the natural history of the inflammatory bowel diseases.

Accordingly, those skilled in the art continue with research and development efforts in the field of IBD.

### SUMMARY OF THE INVENTION

Embodiments of the present invention are generally directed to compounds and compositions for targeting macrophages, which may include a mannan or dextran-based molecular entities in exemplary embodiments. The present invention also includes methods of formulating such compounds and compositions. The present invention also includes methods of treatment using such compounds and compositions.

In one aspect, the present invention provides compounds, compositions and methods for the treatment and management of gastrointestinal diseases (GID) mediated by such cluster differentiating marker expressing macrophages using synthetic and natural macromolecules (e.g., about 1-1000 kDa). These diseases may include any condition where macrophages or other cluster differentiating high-expressing cells are involved or recruited, such as those where the number of macrophages or other cluster differentiating high-expressing cells is increased and/or such cells are metabolically abnormal, such as, but not limited to, in viral infections, altered GI environments, combinations thereof, or the like. Such diseases may include immune diseases, inflammatory diseases, and infectious diseases to name a few examples.

In another aspect, the present invention provides compounds, compositions and methods for the management and/or treatment of diseases mediated by non-macrophages with lectin binding properties.

In exemplary embodiments, a method of treatment may include administering, to a subject afflicted with a GI disease or disorder, a compound comprising a C-type lectin receptor targeting moiety. The method may further include delivering to cells expressing C-type lectin receptors within the subject(s) GI tract, at least one or a combination of (a) therapeutic agent(s) and (a) detection moiety (ies).

In exemplary embodiments, a method of diagnosing may include identification of macrophages in the bowel.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

FIG. 1 is an illustration of the family of C-type Lectin Receptors;

FIG. 2 is the chemical structure of linear mannan;

FIG. 3 is the chemical structure of branched mannan;

FIG. 4 is the chemical structure of galacto-mannan;

FIG. 5 is the chemical structure of gluco-mannan;

FIG. 6 is the chemical structure of galacto-gluco-mannan;

FIG. 7 is the chemical structure of xylo-mannan;

FIG. 8 is an illustration of alternative mannan moieties including phosphorylation;

FIG. 9 is an illustration of Synthetic Route A for the modification of mannose, mannan, and/or mannan congeners;

FIG. 10 is an illustration of Synthetic Route B for the modification of mannose or mannan derivatives;

FIG. 11 is an illustration of the conjugation of doxorubicin to mannose or mannan derivatives;

FIG. 12 is an illustration of the conjugation of amine-containing therapeutic agents;

FIG. 13 is a table of the minear profile of soy protein hydrolysate;

FIG. 14 is a table of the amino acid profile of soy protein hydrolysate;

FIG. 15 is a table of the molecular weight partition of soy protein hydrolysate;

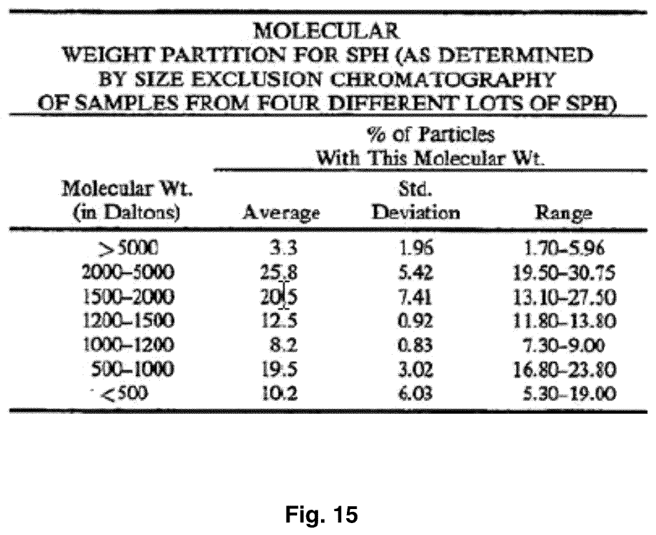

FIG. 16 is a table of the amino acid profile of casein; and

FIG. 17 is a table of general properties of casein.

### DETAILED DESCRIPTION

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein may be made without departing from the scope and spirit of the present invention. In addition, descriptions of well-known functions and constructions are omitted for clarity and conciseness.

### Terminology

As used herein, nomenclature for compounds, including organic compounds, may be referred to using common names, such as but not limited to, those provided in the IUPAC, IUBMB, or CAS recommendations for nomenclature. When one or more stereochemical features are present, Cahn-Ingold-Prelog rules for stereochemistry may be employed to designate stereochemical priority, E/Z specification, and the like. One of skill in the art may readily ascertain the structure of a compound if given a name, either by systemic reduction of the compound structure using naming conventions, or by commercially available software, such as CHEMDRAW™ (available from Perkin Elmer Corporation, U.S.A.).

As used in the specification, the singular forms “a,” “an” and “the” may include plural referents unless the context clearly dictates otherwise. Thus, for example, reference to “a functional group,” “an alkyl,” or “a residue” may include mixtures of two or more such functional groups, alkyls, or residues, and the like.

Ranges may be expressed herein as from “about” one particular value, and/or to “about” another particular value. When such a range is expressed, a further aspect may include the one particular value and/or to the other particular value. Similarly, when values are expressed as approximations, by use of the antecedent “about,” it will be understood that the particular value forms a further aspect. It will be further understood that the endpoints of each of the ranges may be significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 may also be disclosed.

References in the specification to parts by weight of a particular element or component in a composition may denote the weight relationship between the element or component and any other elements or components in the composition or article for which a part by weight is expressed. Thus, in a compound containing two parts by weight of component X and 5 parts by weight component Y, X and Y are present at a weight ratio of 2:5 and are present in such ratio regardless of whether additional components are contained in the compound.

A weight percent (wt. %) of a component may be based on the total weight of the formulation or composition in which the component is included.

As used herein, the terms “optional” or “optionally” may refer to the subsequently described event or circumstance may or may not occur, and that the description may include instances where said event or circumstance occurs and instances where it does not. The omission of “optional” or “optionally” does not necessarily mean that the subsequently described event, circumstances, or components are mandatory.

As used herein, the term “subject” may include a vertebrate, such as a mammal, a fish, a bird, a reptile, or an amphibian. Thus, the subject of the herein disclosed methods may include a human, non-human primate, horse, pig, rabbit, dog, sheep, goat, cow, cat, guinea pig or rodent. The term does not denote a particular age or sex. Thus, adult, and newborn subjects, as well as fetuses, whether male or female, are intended to be covered. In one aspect, the subject is a mammal. A patient may refer to a subject afflicted with a disease or disorder. The term “patient” may include human and veterinary subjects.

As used herein, the term “treatment” may refer to the medical management of a patient with the intent to cure, ameliorate, stabilize, mitigate progression, or prevent a disease, pathological condition, or disorder. This term may include active treatment, e.g., treatment directed specifically toward the improvement of a disease, pathological condition, or disorder, and also may include causal treatment, e.g., treatment directed toward removal of the cause of the associated disease, pathological condition, or disorder. In addition, this term may include palliative treatment, e.g., treatment designed for the relief of symptoms rather than the curing of the disease, pathological condition, or disorder; preventative treatment, e.g., treatment directed to minimizing or partially or completely inhibiting the development of the associated disease, pathological condition, or disorder; and supportive treatment, e.g., treatment employed to supplement another specific therapy directed toward the improvement of the associated disease, pathological condition, or disorder. In various aspects, the term covers any treatment of a subject, including a mammal (e.g., a human), and may include: (i) preventing the disease from occurring in a subject that may be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also may include domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” may refer to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “modulate” or “regulate” referring to cells may induce altered states of the cell or cells as this may relate to their phenotype or production of factors, e.g. IL-6, interferon, cytokines or other gene or cell product expression.

As used herein, the term “diagnosed” may include having been subjected to a physical examination by a person of skill, for example, a physician, and found to have a condition that may be diagnosed or treated by the compounds, compositions, or methods disclosed herein.

As used herein, the phrase “identified to be in need of treatment for a disorder,” or the like, may refer to selection of a subject based upon need for treatment of the disorder. For example, a subject may be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the identification may be performed by one who subsequently performed the administration.

As used herein, the terms “administering,” and “administration” refer to any method of providing a pharmaceutical preparation to a subject. Such methods are well known to those skilled in the art and may include, but are not limited to, oral administration, rectal administration, sublingual administration, buccal administration (oral pouch), and enteral administration, the latter by voluntary ingestion, or mechanical administration. Administration may be continuous or intermittent. In various aspects, a preparation may be administered therapeutically, e.g., administered to treat an existing disease or condition. In further various aspects, a preparation may be administered prophylactically, e.g., administered for prevention of a disease or condition. Combinations of administration may be utilized.

The term “contacting” as used herein may refer to bringing a disclosed compound and a cell, a target receptor (e.g. CD206, CD209, CD69 or other C-type lectin receptor or a receptor expressed by macrophage cell type(s) or other receptor or cell type which may bind a mannan-based construct), or other biological functional component or entity together in such a manner that the compound may affect the activity of the target, either directly; i.e., by interacting with the target itself, or indirectly; i.e., by interacting with another molecule, co-factor, factor, cell type or protein on which the activity of the target is dependent.

As used herein, the terms “effective amount” and “amount effective” may refer to an amount that is sufficient to achieve the desired result or to have an effect on an undesired condition. For example, a “therapeutically effective amount” may refer to an amount that is sufficient to achieve the desired therapeutic outcome or to have an effect on undesired symptoms but is generally insufficient to cause unacceptable adverse side effects. The specific therapeutically effective amount (i.e., dose level) for any particular patient may depend upon a variety of factors including, but not limited to, the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed; combinations thereof; and like factors well known in the medical arts. For example, it is well within the skill of the art to start doses of a compound at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. The dosage may be adjusted by the individual physician in the event of any contraindications. Dosage may vary, and may be administered in one or more dose administrations daily, for one or several days. Guidance may be found in the literature for appropriate dosages for given classes of pharmaceutical products. In further various aspects, a preparation may be administered in a “prophylactically effective amount;” e.g., an amount effective for prevention of a disease or condition.

The term “pharmaceutically acceptable” describes a material that is not biologically or otherwise undesirable, e.g., without causing an unacceptable level of undesirable biological effects or interacting in a deleterious manner.

As used herein, the term “pharmaceutically acceptable carrier” may refer to sterile aqueous or nonaqueous solutions, dispersions, suspensions, or emulsions, as well as sterile powders for reconstitution into sterile medically desirable solutions or dispersions just prior to use. Examples of suitable aqueous and nonaqueous carriers, diluents, solvents or vehicles include but are not limited to water, polyols (e.g., glycerol, propylene glycol, polyethylene glycol and the like), carboxymethylcellulose and suitable mixtures thereof, vegetable/plant oils (such as olive oil), animal oils (e.g., fish oil and fish oil derivatives), vitamins, protein and protein constructs (either natural or synthetic), peptides. Proper fluidity or viscosity may be maintained, for example, by the use of materials such as lecithin, and by the maintenance of the required particle size in the case of dispersions and by the use of surfactants. These compositions may also contain adjuvants such as preservatives, wetting agents, emulsifying agents, chelation agents and dispersing agents. Prevention of the action of microorganisms may be ensured by the inclusion of various antibacterial and antifungal agents such as sorbic acid and the like where such preservatives are approved for delivery into the G.I. tract. It is additionally desirable to include isotonic agents such as sugars, sodium chloride and the like. Prolonged absorption of the delivered pharmaceutical form may be brought about by the inclusion of agents, such as gelatin or non-digestible fiber, which delay absorption. Polymers such as polylactide-polyglycolide, poly(orthoesters) and poly(anhydrides). Depending upon the ratio of other ingredients to polymer and the nature of the particular polymer employed the rate of release may be controlled. Formulations are also prepared by entrapping the mannan or mannan constructs or mannan derivatives in liposomes or microemulsions which are compatible with body tissues. The formulations may be sterilized, for example, by pasteurization or other acceptable non-degrading means of removing potentially harmful matter or organisms. Suitable inert carriers may be added to the formulation to control palatability and digestibility and may include sugars such as glucose, sucrose, or other acceptable flavoring agent, such as saccharin, vanilla, cinnamon, etc. Desirably, at least 95% by weight of the mannan or mannan derivatives have an effective molecular weight size in the range of 100 Da (mannose) to 200,000 Da (chains and chain derivatives). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon may be depicted as a wedge (bonds to atoms above the plane) and the other may be depicted as a series or wedge of short parallel lines is (bonds to atoms below the plane). The Cahn-Inglod-Prelog system may be used to assign the (R) or (S) configuration to a chiral carbon.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that may be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that may be performed it is understood that each of these additional steps may be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that may perform the same function that are related to the disclosed structures, and that these structures will typically achieve the same result.

### Inflammatory Bowel Disease

Macrophages are a type of white blood cell of the innate immune system that engulfs and digests pathogens. Macrophages are found in essentially all tissues and are known to take various forms throughout the body. These include, for example, Kupffer cells of the liver, mesangial cells of the kidney, and various types of macrophages of the gastrointestinal tract (such as mucosal and intraepithelial macrophages).

Macrophages can exhibit different phenotypes in response to various stimuli, resulting in the expression of different surface markers, receptors, and proteins. A type of receptor expressed by macrophages are C-type lectin receptors such as CD206, CD207, CD209, CD210a, CD210b, CD147, CD299, etc.

Macrophages can be considered gatekeepers of intestinal immune homeostasis since they discriminate between innocuous antigens and potential pathogens to maintain oral tolerance. However, in some individuals with a genetic and environmental predisposition, regulation of intestinal immunity may be impaired, leading to chronic relapsing immune activation and pathologies of the gastrointestinal tract, such as IBD.

There is evidence to suggest a causal link between defects in the resolution of intestinal inflammation and altered monocyte-macrophage differentiation in patients with IBD. Due to this, macrophages are a novel potential target to develop new treatment approaches. The question then is how to best achieve altered states of these resonant macrophages which consist of multiple intermediate populations residing between two effective main classes—i.e., pro- and anti-inflammatory populations of macrophages.

It has been observed that gastrointestinal macrophages appear to behave similarly to peritumoral macrophages, the latter expressing a host of C-type lectin receptors which can be effectively exploited for diagnostic purposes in breast cancer, melanoma, and squamous cell carcinoma.

Specifically targeting the lectin receptor CD206, and other related lectin receptors with high mannose recognition sites, enables those lectin receptors to be exploited to alter inflammatory macrophage phenotypes and, in turn, transition to less inflammatory or noninflammatory states.

C-type lectin receptors expressed by macrophages and other immune cells can be exploited in such a manner as to alter their phenotypic states. The alteration of such states may result in a shift in their immune functions by either enhancing or dampening their activities and interactions within immune cell complexes. Such an approach to targeting macrophages and the immune cells that express C-type lectin receptors (i.e., altering their functional states in a manner which either enhances or mitigates a desired functionality relative to existing normal states or pathologies) is a novel unrecognized approach to mitigating inflammatory states in gastrointestinal diseases.

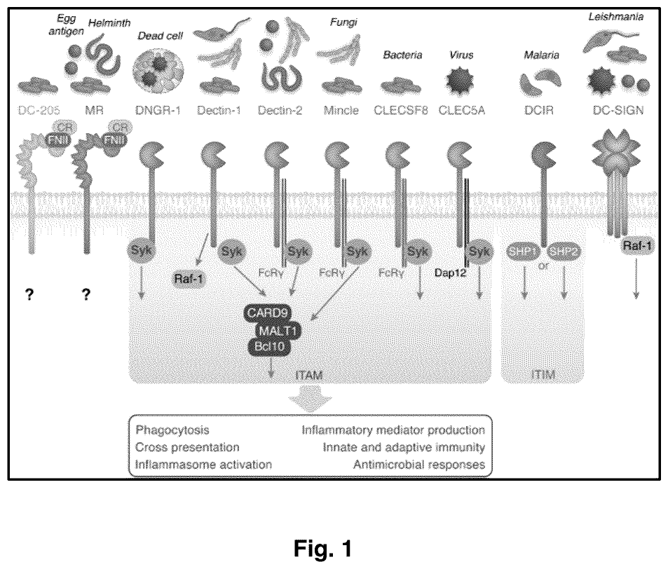

C-type lectin receptors are evolutionarily derived as recognition elements for invading pathogens. These receptors (FIG. 1) are adapted to recognize specific carbohydrate moieties located on the surfaces of pathogenic organisms, including bacteria, viruses, as well as free antigens bearing carbohydrate-inducing immune and or other cell signal responses. Recognizing carbohydrate moieties on invading pathogens (a key part of this process) results in an immune cascade, activating multiple cell types of the immune system as a matter of dealing with mitigation of the pathogen-induced disease process. However, this process can go awry, leading to chronic conditions such as autoimmune diseases, or overreaction of the immune system to invading pathogens. In some cases, attempts are made to suppress an overwrought immune system, such as in the case of extraordinary inflammation diseases like rheumatoid arthritis, Crohn's disease, ulcerative colitis, and bowel diseases involving disease-driving C-type lectin receptors. It is not uncommon to treat the inflammation element of these diseases with immunosuppressive steroids or nonsteroidal anti-inflammatory drugs. However, these approaches are fraught with significant side effects, especially when these drugs are used long-term.

It is understood that cells (especially immune cells) exist in a span of phenotypes, all of which may be modulated and many of which can be interchanged. It is possible to identify compounds, molecules, classes of chemistry, etc. that may be used to effect such phenotypic changes or enter phenotypic changes. Macrophages are known to exist in two major states (“pro-inflammatory” and “anti-inflammatory”) with multiple interface states. In the case of inflammatory bowel diseases, macrophages and other immune cells exist in a state of response, a pro-inflammatory state. This phenotypic state results in the production of multiple chemokines, cytokines, and proteinaceous activators that affect the entire gastrointestinal macroenvironment and microenvironments.

A key approach to managing gastrointestinal inflammatory diseases is to modulate pro-inflammatory states of cells that participate in the natural history and progression of the disease. One such candidate would be any pseudo-ligand of C-type lectin receptors.

C-type lectin receptors have the potential to accept high mannose constructs that, when flooded into the gastrointestinal environment of patients suffering from inflammatory bowel diseases, may shift C-type lectin receptor bearing cells to altered states of low inflammatory or non inflammatory status.

### Formulations

The present invention relates to formulations containing a C-type lectin receptor ligand capable of achieving altered states in pro-inflammatory macrophages of the gastrointestinal tract, thereby providing a functional clinical approach to the mitigation of gastrointestinal inflammation which may be categorically and clinically assessed as Crohn's disease, inflammatory bowel syndrome, ulcerative colitis, and other gastrointestinal disorders in which macrophages play a central element of the natural history of the disease. It is contemplated that said formulations represents a wholly unique methodology in approaching gastrointestinal diseases.

In some embodiments, the process for treating IBD and other diseases/disorders involve the oral delivery of a dietary formulation containing either singly or a mixture of mannan congeners.

As used herein, the term “mannan congeners” refers to mannan itself (mannose-based polysaccharide) and other structurally related compounds (including other polysaccharides) that are similar in backbone or composition to mannan but may differ in various ways—e.g., branching, linkage types, substitutions, etc.

The congeners may include, but are not limited to, straight chain or branched chain mannan (poly-mannose), straight or branched galactomannan, straight chain or branched glucomannan, and/or straight or branched xylo-mannan or other mannan congeners that can: (a) effectively bind to lectin-type receptors of macrophages that are functionally involved in bowel diseases; and (b) alter their phenotypic states such that they are less inflammatory or noninflammatory. The mannan congener(s) may be utilized in any suitable proportion to achieve desirable clinical outcomes.

In some embodiments, the dietary formulation is provided in liquid form enriched with dietary nutrients such that the formulation can be a meal supplement or replacement. In some embodiments, the dietary formulation may be delivered enterally.

In some embodiments, the formulation contains eicosapentaenoic acid (20:Sn3) and/or docosahexaenoic acid (22:6n3), and a source of lectin receptor binding mannan constructs and/or derivatives with a molecular weight range of between 100 Da and 200,000 Da which effectively mitigates the contribution of pathological inflammatory macrophages in the natural history of inflammatory bowel diseases via a shift in the saturation of C-type lectin receptor saturation. Preferably the nutritional product also contains one or more nutrients which act as antioxidants.

### 1. Mannan Congeners

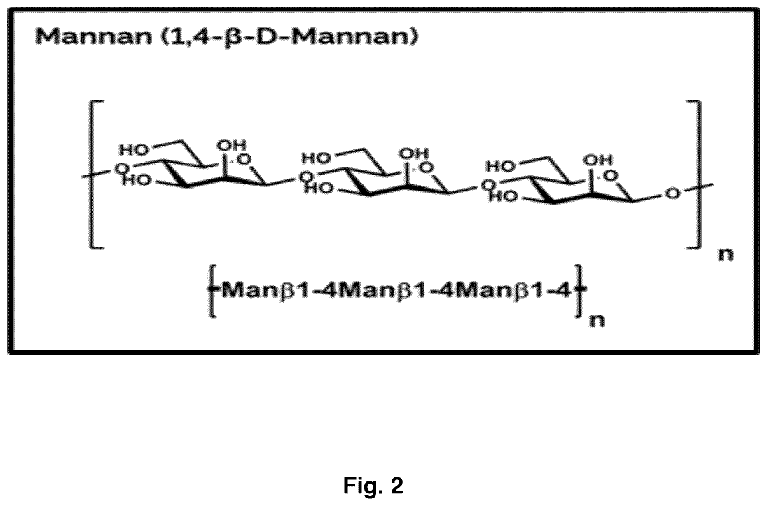

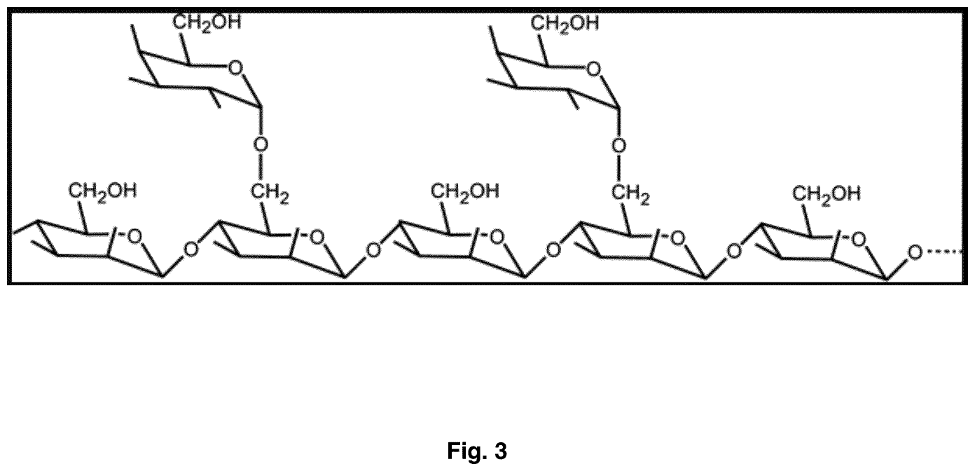

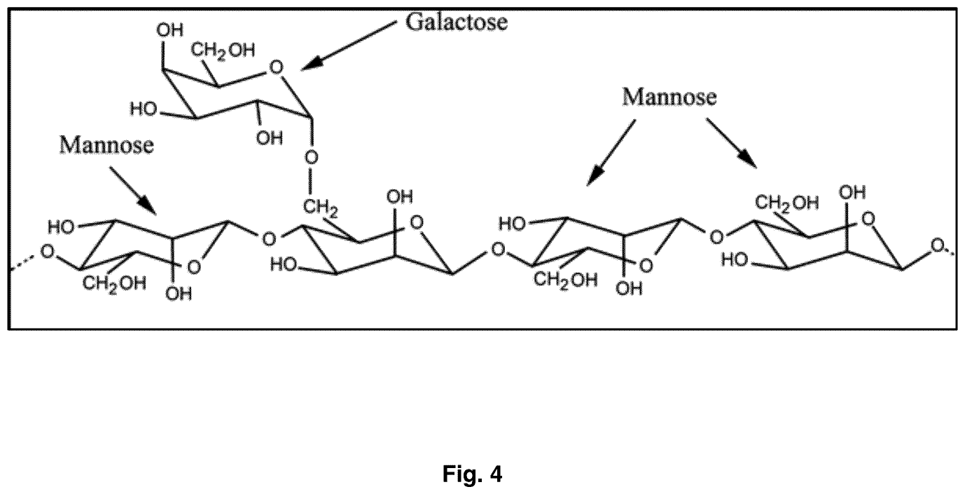

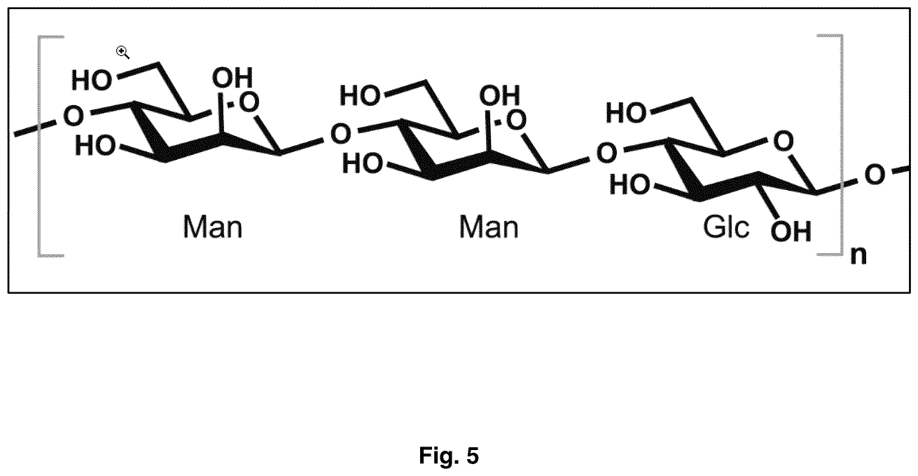

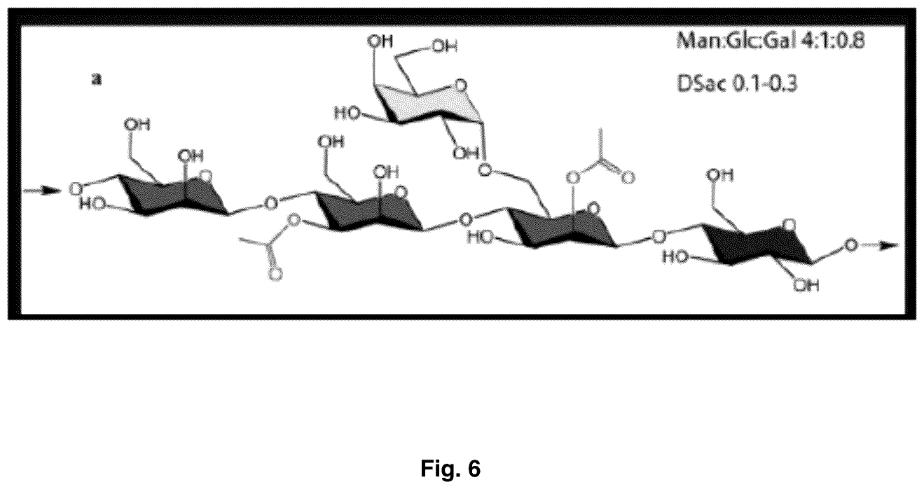

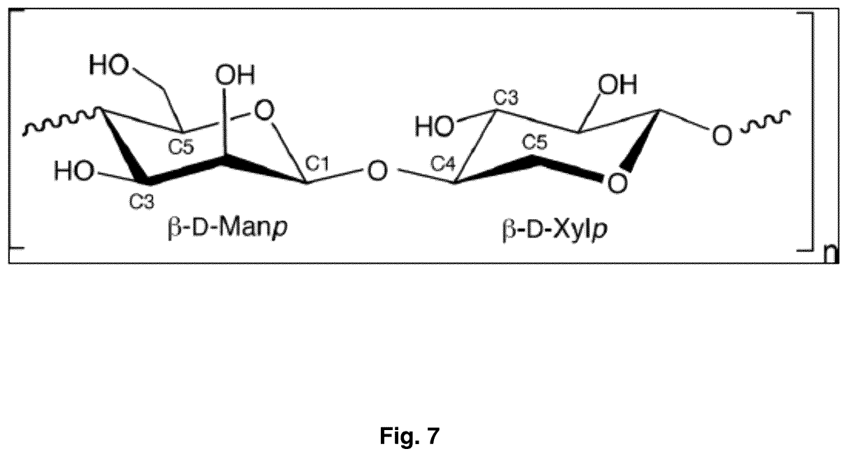

Examples of mannan congeners which may be suitable in embodiments of the present invention include without limitation, linear or branched mannan, linear or branched galactomannan, linear or branched glucomannan, and all such forms thereof. FIGS. 2-7 provide examples of certain mannan congeners which may be suitable for use in formulations according to the present invention.

As a general rule of molecular transmembrane transport, constructs having smaller molecular weight (MW) may be appropriate for instances where the molecule is desired reduce resistance to transport barriers, or when reduced residence time is desired (i.e., the duration of binding to C-type lectin receptors is reduced). This principle may be independent of characteristics such as the charge of the molecule, tertiary folding anomalies, reaction affinities, or other molecular-specific characteristics. Constructs having larger MW may be appropriate for instances where increased residence time is desired (i.e., the duration of binding to CD206 is increased). In certain embodiments, constructs having smaller MW (e.g., up to about 200,000 Da) may be employed when more efficient receptor substrates are attached to the backbone (e.g., branched mannose moieties). More efficient receptor substrates may bind to C-type lectin receptors for longer durations and/or more effectively, thus allowing for the use of smaller mannose or mannan constructs.

Preferably, the formation includes a source of mannan or mannan derivatives where the mannan or mannan derivatives, either alone or in combination, have a molecular weight distribution of 100 Da to 200,000 Da, and wherein the mannan and/or derivatives comprise between 1% and 20% of the dry weight of the final ingredient list (dry ingredients in toto), or more preferably between 2% and 10%, or even more preferably between 4% and 8%.

In some embodiments, the formulation may include a quantity of mannans derived from yeast. The molecular weight distribution of mannans and derivatives found in yeast generally ranges from 100 Da (mannose) to 400 kDa. Commonly available forms of yeast mannans include YM50, YM70, AND YM90, which have molecular weights of 173, 87, and 54 kDa, respectively.

In some embodiments, combinations or mixtures of different forms of yeast mannans may be used to achieve a desired property in the formulation, such as a desired weight addition or viscosity (such that it would be clinically acceptable for oral or tube delivery). In preferred embodiments, the quantity of mannans and derivatives used in the formulation would represent a weight addition ranging from about 10 g/L to about 30 g/L, or more preferably from about 16 g/L to about 24 g/L, or even more preferably about 20 g/L (i.e., about 2% w/v). The table below lists some preferred specifications for the yeast mannans.

| ITEMS | SPECIFICATION | | RESULTS |
|---|---|---|---|
| ASSAY | ≥92% | | 95.2% |
| Appearance | White Powder | | Complies |
| Odor | Odorless | | Complies |
| Water Solubility | Completely Soluble in Water | | Complies |
| Loss on Drying | ≤5% | | 4.4% |
| Ash | ≤5% | | 4.4% |
| As | ≤1 | ppm | Complies |
| Pb | ≤1 | ppm | Complies |
| Cd | ≤1 | ppm | Complies |

Microbiological

| Total Plate Count | ≤2000 | CFU/g | Complies |
|---|---|---|---|
| Yeast/Mold | ≤125 | CFU/g | Complies |
| Salmonella | Neg. | | Neg. |
| Thermo-tolerant | Neg. | | Neg. |
| Coliform | | | |

| | | |
|---|---|---|
| Staph | Neg. | Neg. |

Note:

| Storage | Store at 6 to 18 Degrees Celsius in a low humidity atmosphere |
|---|---|
| Shelf Life | 2 years - if sealed and stored from direct sunlight and as above |
| Molecular Weight | Molecular weight distribution may range from 100 Da (mannose) |
| Distribution | to 400 kDa. Typical molecular weights are the forms YM50, |
| | YM70, AND YM90, with molecular weights of 173, 87, and 54 |
| | kDa, respectively. Molecular weight mixtures will be used to |
| | achieve a desired viscosity for clinically acceptable oral or tube |
| | delivery, but also achieve a weight addition of 20 g/L or about 2% |
| | w/v with a range of 10 g/L to 30 g/L, and a preferred range of |
| | 16 g/L to 24 g/L. |
| Other Notes | L = liter; CFU = colony forming units; ppm = parts per million; Neg = |
| | negative; g = gram weight |

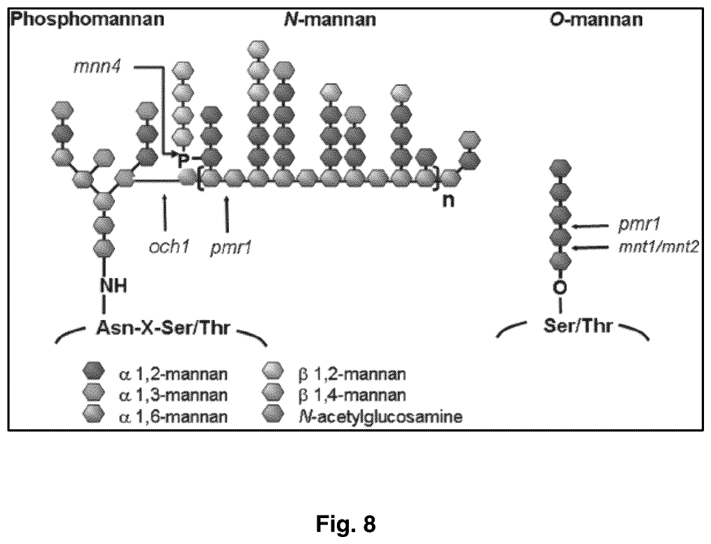

In some embodiments, the mannan congeners may be chemically modified, such as by the conjugation of various moieties (the mannan congener acting as a carrier molecule)—e.g., C-type lectin receptor targeting moieties, therapeutic agents, and detection labels. The mannan congener(s) may also include (in addition to carbohydrate moieties other than mannan, galactose, glucose or xylose), lipid and/or amino acid and/or nucleic acid (monomers, oligomers and polymers; mono-amino acids, dipeptides, oligo-peptides, proteins; monomer nucleotides/nucleosides, oligo-nucleotides/nucleosides, polymeric nucleotides/nucleosides) moieties and other chemical modifications as may prove efficacious, e.g., PO4-3 (phosphorylation) (e.g., FIG. 8).

### A. Targeting Moiety

In some embodiments, a C-type lectin receptor targeting moiety is included. The C-type lectin receptor targeting moiety may be include, but is not limited to, mannose, glucose, galactose, xylose, fucose, and/or n-acetylglucosamine. In some embodiments, the targeting moieties are attached to between about 5% and about 75% of the backbone residues or between about 20% and about 45% of the residues, or between about 15% and about 40% of the residues. It is noted that the MWs referenced herein, as well as the number and degree of conjugation of receptor substrates, linkers, and diagnostic/therapeutic moieties attached may refer to average amounts for a given quantity, since the production and purification techniques will result in some variability.

### B. Therapeutic Agent

In some embodiments, the process for treating IBD and other diseases/disorders do not involve the use of antibodies, anti-idiotypic antibodies, or currently categorized drugs.

In other embodiments, it may be desirable to not only shift the inflammatory status of gastrointestinal resident macrophages, but rather to eliminate them entirely from the gastrointestinal environment. Thus, attachment of therapeutic agents to achieve this result to be considered therapeutically necessary, would involve the chemical linking of agents to the mannan congener backbone such as may achieve, by binding to C-type lectin receptors, localized induction of apoptosis in gastrointestinal resident macrophages.

Therapeutic agents may include, but are not limited to, chemotherapeutic agents, such as doxorubicin; drugs with effect on immune cells; anti-infective agents, such as antibiotics (e.g. tetracycline, streptomycin, amphotericin and isoniazid), heavy metals such as antimony (e.g. pentavalent antimonials), anti-virals, anti-fungals, and anti-parasitics; immunological adjuvants; steroids; nucleotides, such as DNA, RNA, RNAi, siRNA, CpG or Poly(I:C); peptides; proteins; or metals such as silver, gallium or gadolinium, paromomycin, miltefosine, fluconazole, pentamide, Meglumine antimoniate, and combinations thereof.

In certain embodiments, the therapeutic agent is an antimicrobial drug selected from the group comprising: an antibiotic; an anti-tuberculosis antibiotic (such as isoniazid, streptomycin, or ethambutol); drugs which effect Corona viruses and other RNA viruses; an anti-viral or anti-retroviral drug, for example an inhibitor of reverse transcription (such as zidovudine) or a protease inhibitor (such as indinavir). In certain embodiments, the therapeutic agent is an anti-microbial active, such as amoxicillin, ampicillin, tetracyclines, aminoglycosides (e.g., streptomycin), macrolides (e.g., erythromycin and its relatives), chloramphenicol, ivermectin, rifamycins and polypeptide antibiotics (e.g., polymyxin, bacitracin) and zwittermicin. In certain embodiments, the therapeutic agent is selected from isoniazid, doxorubicin, streptomycin, and tetracycline.

In some embodiments, the therapeutic agent comprises a high energy killing isotope which has the ability to kill macrophages and tissue in the surrounding macrophage environment. Suitable radioisotopes include: ^{210/212/213/214}Bi, ^{131/140}Ba, ^{11/14}C, ⁵¹Cr, ^{67/68}Ga, ¹⁵³Gd, ^{99m}Tc, ^{88/90/91}Y, ^{123/124/125/131}I, ^{111/115m}In, ¹⁸F, ¹⁰⁵Rh, ¹⁵³Sm, ⁶⁷Cu, ¹⁶⁶Ho, ¹⁷⁷Lu, ^{186/188} Re, ^{32/33}P, ^{46/47}Sc, ^{72/75}Se, ³⁵S, ¹⁸²Ta, ^{123m/127/129/132}Te, ⁶⁵Zn and ^{89/95}Zr.

In other embodiments, the therapeutic agent comprises a non-radioactive species selected from, but not limited to, the group consisting of: Bi, Ba, Mg, Ni, Au, Ag, V, Co, Pt, W, Ti, Al, Si, Os, Sn, Br, Mn, Mo, Li, Sb, F, Cr, Ga, Gd, I, Rh, Cu, Fe, P, Se, S, Zn and Zr, where these are primarily divalent cations.

In still further embodiments, the therapeutic agent is selected from the group consisting of modulating agents, antivirals, cytostatic agents, cytocidal agents alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radio-sensitizing drugs. By way of more specific example, the therapeutic agent is selected from the group consisting of mechlorethamine, triethylenephosphoramide, cyclophosphamide, ifosfamide, chlorambucil, busulfan, melphalan, triaziquone, nitrosourea compounds, Adriamycin, carminomycin, daunorubicin (daunomycin), doxorubicin, isoniazid, indomethacin, gallium (III), 68gallium (III), aminopterin, methotrexate, amethopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, Taxol, taxane, cytochalasin B, gramicidin D, ethidium bromide, emetine, tenoposide, colchicin, dihydroxy anthracin Dione, mitoxantrone, procaine, tetracaine, lidocaine, propranolol, puromycin, ricin subunit A, abrin, diphtheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, hydroxychloroquine (or analogs), remdesivir (or analogs) esperamicins, and dynemicins.

In embodiments wherein the therapeutic agent is a hormone or hormone antagonist, the therapeutic agent may be selected from the group consisting of prednisone, hydroxyprogesterone, medroprogesterone, diethylstilbestrol, tamoxifen, testosterone, and aminogluthetimide.

In embodiments wherein the therapeutic agent is a prodrug, the therapeutic agent may be selected from the group consisting of phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that may be converted to the more active cytotoxic free drug.

### C. Detection Labels

Diagnostic and therapeutic methods are disclosed for the in vivo modulation, eradication (i.e., lethality), and/or detection of diseases or conditions using the disclosed compounds. The overall construct of the mannan congener backbone with C-type lectin receptor targeting moieties may be used for these purposes without added carrying moieties, chelation moieties, nor the chelation of cations where there is intent to therapeutically modulate macrophage states or expression. However, certain moieties may be added such that the modulation effect is retained while still providing for diagnostic purposes as well, making the overall schema bimodal.

In certain embodiments, the disclosed compounds may include a detection label in addition to the therapeutic agent. As used herein, the term “detectable label or moiety” may refer to an atom, isotope, or chemical structure that is: (1) capable of attachment to the carrier molecule; (2) non-toxic to humans or other mammalian subjects; and (3) provides a directly or indirectly detectable signal, particularly a signal that is not only measurable, but whose intensity is related (e.g., proportional) to the concentration of the detectable moiety. The signal may be detected by any suitable method of detection, including spectroscopic, electrical, optical, magnetic, auditory, radio signal, or palpation detection.

Detection labels include, but are not limited to, fluorescent molecules (a.k.a. fluorochromes and fluorophores), chemiluminescent reagents (e.g., luminol), bioluminescent reagents (e.g., luciferin and green fluorescent protein (GFP)), metals (e.g., gold nanoparticles), and radioactive isotopes (radioisotopes). Suitable detection labels may be selected based on the choice of imaging method. For example, the detection label may be a near infrared fluorescent dye for optical imaging, a Gadolinium chelate for MRI imaging, a radionuclide for PET or SPECT imaging, or a gold nanoparticle for CT imaging.

Detection labels may be selected from, for example, a radionuclide, a radiological contrast agent, a paramagnetic ion, a metal, a fluorescent label, a chemiluminescent label, an ultrasound contrast agent, a photoactive agent, or a combination thereof. Non-limiting examples of detectable labels include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn ⁵⁵Co, ⁷²As, ⁷⁵Br, ⁷⁶Br, ^{82m}Rb, ⁸³Sr, ^{117m}Sn or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas-filled liposomes.

Other suitable labels include, for example, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), near IR dyes, quantum dots, phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that may be detected using NMR or ESR spectroscopy. Such labeled molecules may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label. Another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strep) avidin binding pair. Such a functional group may be used to link a disclosed compound to a protein, polypeptide or chemical compound that is bound to the other half of the binding pair, i.e., through formation of the binding pair. For example, such a conjugated molecule may be used as a reporter, for example, in a diagnostic system where a detectable signal-producing agent is conjugated to avidin or streptavidin.

### D. Chelating Agents

In some embodiments, a chelating agent may be attached or incorporated into a disclosed compound, and used to chelate a therapeutic or diagnostic agent, such as a radionuclide. Exemplary chelators include but are not limited to DTPA (such as Mx-DTPA), DOTA, TETA, NETA or NOTA. Other useful chelators include, but are not limited to, DTPA, DO3A, DOTA, EDTA, TETA, EHPG, HBED, NOTA, DOTMA, TETMA, PDTA, TTHA, LICAM, HYNIC, and MECAM. HYNIC is particularly useful for chelating technetium-99 (Tc99), another imaging agent of the invention.

### E. Synthesis

The compounds of this invention may be prepared by employing reactions as shown in the disclosed schemes, in addition to other standard manipulations that are known in the literature, exemplified in the experimental sections or clear to one skilled in the art. The following examples are provided so that the invention might be more fully understood, are illustrative only, and should not be construed as limiting. For clarity, examples having fewer substituents may be shown where multiple substituents are allowed under the definitions disclosed herein.

It is contemplated that each disclosed method may further comprise additional steps, manipulations, and/or components. It is also contemplated that any one or more step, manipulation, and/or component may be optionally omitted from the invention. It is understood that a disclosed method may be used to provide the disclosed compounds. It is also understood that the products of the disclosed methods may be employed in the disclosed compositions, kits, and uses.

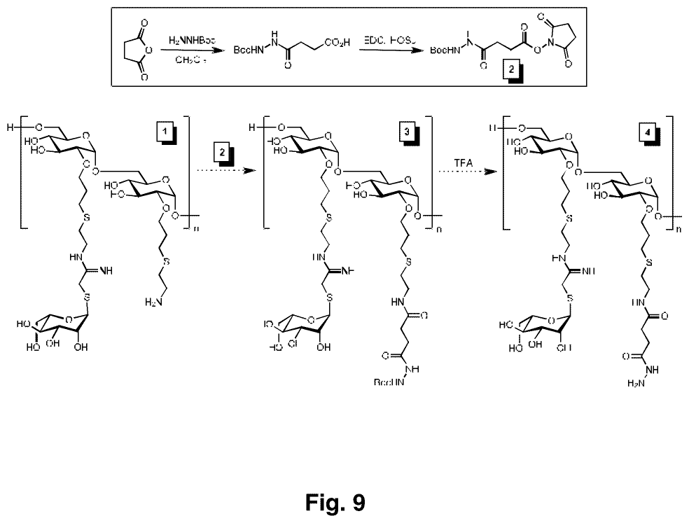

The compounds of the present invention may be synthesized by any number of ways known to one of ordinary skill in the art. Referring to Scheme 1 (FIG. 9), for example, linker 2 may be synthesized by initiating a ring opening reaction of succinic anhydride with tert-butyl carbazate. The resulting intermediary comprises a carboxyl end group and a tert-Butyloxycarbonyl end group (for protection). The carboxyl may then be activated by EDC coupling reagent to form an ester linkage with N-hydroxy succinimide (NHS), yielding linker 2. Linker 2 may then be functionalized onto mannose, mannan or mannan congener (e.g., 1-3 of Scheme 1) through an amide linkage by replacing NHS. Then, the Boc protecting group may be removed under dilute acidic condition (typically 30-40% trifluoroacetic acid in DMSO) to obtain 4. Dilute acidic condition may be required to avoid any unwanted cleavage of the glycosidic linkage present in the backbone. The resulting functionalized construct may be purified by size exclusion filtration.

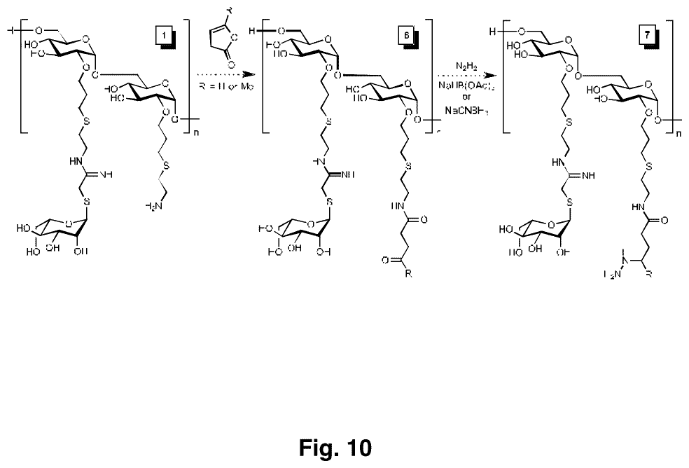

Alternatively, compounds according to the present invention may be synthesized according to FIG. 10. Referring to which, as shown below, the free primary groups of mannan may be reacted with an excess of lactone under anhydrous conditions, thereby opening lactone rings and forming amide linkages therebetween. Unreacted lactone may be removed under reduced pressure to obtain modified mannan 6. The corresponding hydrazine derivative 7 may be prepared by reductive amination reaction using sodium cyanoborohydride or sodium triacetoxy borohydride as the reducing agent. The resulting linker terminates in a diimide end group that may be suitable for the attachment of therapeutic agents, detection labels, and the like.

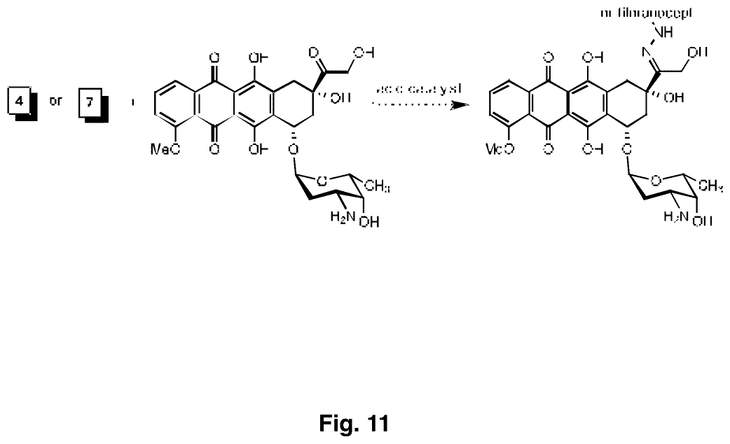

The conjugation of oxo-containing therapeutic agents to mannan derivatives 4 or 7 may be performed as shown in Scheme 3 (FIG. 11). Mannan derivative 4 or 7 may be conjugated to doxorubicin via the diimide end group by the formation of a hydrazone linkage under anhydrous acidic condition or aqueous acidic conditions. Unconjugated therapeutic agent may be removed (e.g. by size exclusion chromatography or dialyzation) to obtain the pure conjugated mannan.

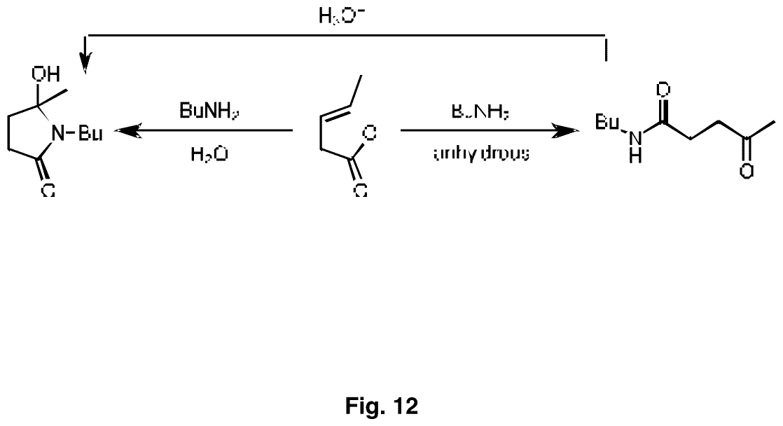

Amine-containing therapeutic agents may be conjugated to the compounds according to FIG. 12. The basic reaction between a primary amine and the lactone are shown in FIG. 12.

### 2. Dietary Nutrients

### A. First Source of Protein

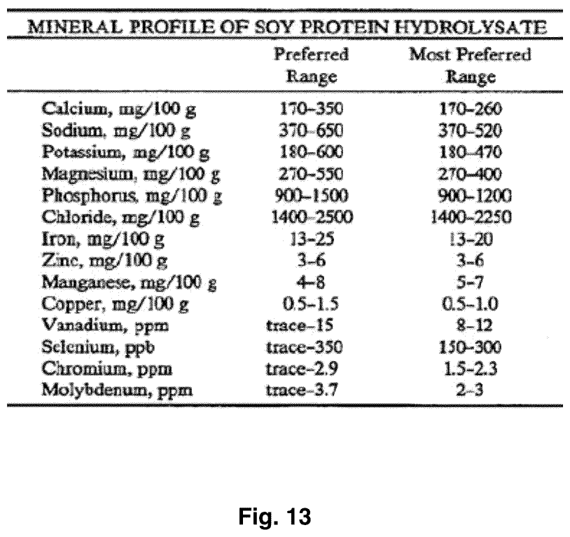

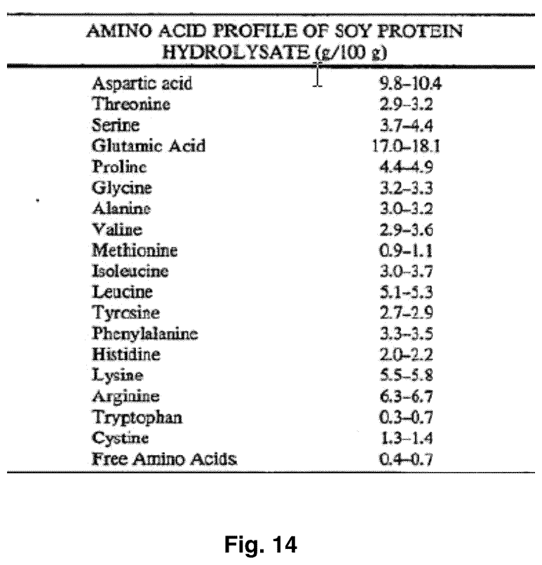

In some embodiments, the nutritional product may include a first source of protein that includes a soy protein hydrolysate. Provided in FIGS. 13-14 is the mineral profile, amino acid profile, and molecular weight partitions of soy protein hydrolysate. Preferably, at least 65% of the soy protein hydrolysate particles have a molecular weight in the range of 700 to 7000 Da. Preferably, the degree of hydrolysis of the soy protein hydrolysate ranges from about 12 to about 20. Preferably, the amino acid profile of the soy protein hydrolysate is less than about 3% free amino acids, or more preferably less than about 2% free amino acids. Preferably, 25% to 65% of the oligopeptides and polypeptides have a molecular weight range of 1200 to 7500 Da.

### B. Second Source of Protein

In some embodiments, the nutritional product may include a second source of protein. The second source of protein may be provided to yield a stable emulsion and/or fill a satisfactory and metabolically appropriate amino acid profile. The second source of protein may include an “essential” amino acid profile including histidine, isoleucine, leucine, lysine, methionine, phenylalanine, threonine, tryptophan, and valine, with each preferably comprising not less than 2.35% of the total protein.

In some embodiments, the second source of protein may include a source of intact protein, which are proteins that are in an original, unaltered form, as it exists naturally in food or biological systems. In preferred embodiments, the source of intact protein may include whey protein isolate, fish protein isolate, potato protein isolate, and/or avian egg albumin isolate.

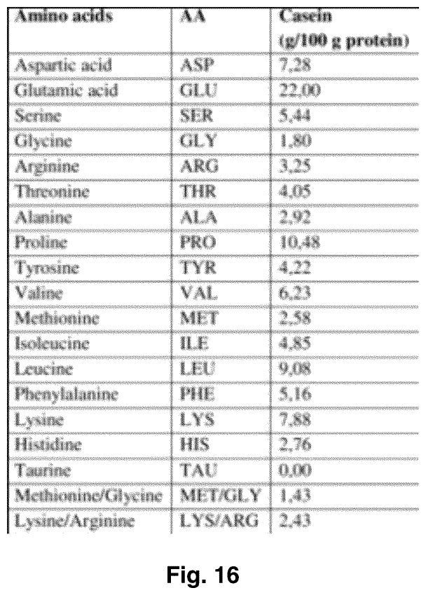

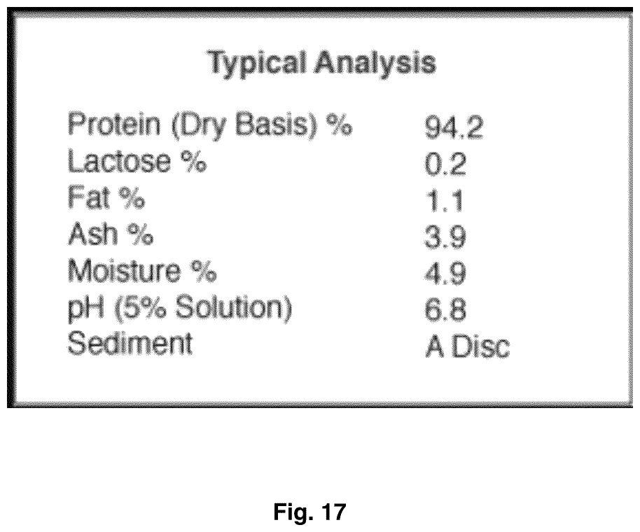

In preferred embodiments, the second source of protein includes casein or a salt thereof such as sodium caseinate. Casein is a major protein found in milk and dairy products that remains largely in its native structure and functional form. Provided in the FIGS. 16-17 are the amino acid profile and general properties of casein. Preferably, the formulation includes sodium caseinate, wherein the sodium caseinate comprises approximately 8% to 28% by weight of the protein in the formulation.

In some embodiments, the second source of protein includes protein hydrolysates, including whey protein hydrolysate and/or hydrolyzed sodium caseinate, preferably selected based on stability.

### C. Source of Fat

In some embodiments, the nutritional product may include a source of fat. Preferably, the source of fat may include polyunsaturated fatty acids such as omega-3 (n-3) and omega-6 (n-6) fatty acids. In some embodiments, the sum of the n-3 fatty acids to n-6 fatty acids approximates 30-45% n-3 to this mixture. In some embodiments, the sum of the n-3 fatty acids to n-6 fatty acids approximates 25-40% n-3 to this mixture. In some embodiments, the source of fat may include fish oil, canola oil, and/or avocado oil. In preferred embodiments, the source of fat may include medium chain triglycerides (i.e., triglycerides with two or three fatty acids having an aliphatic tail of 6-12 carbon atoms).

### 3. Emulsifying Agents

In some embodiments, the nutritional product may include one or more emulsifying agents, which help to stabilize the nutritional product formulation and facilitate uniform mixing. Preferably, the emulsifying agent may include soy lecithin, egg yolk lecithin, and/or sunflower lecithin.

### 4. Other Inclusions

In some embodiments, the formulation includes carotenoids, anthocyanins, and antioxidants.

In some embodiments, the formulation includes (on a per liter basis) an approximate isotonicity balance: (a) 950 to 1100 mg of sodium; (b) 2400 to 2650 mg of potassium; and (c) about 1250 to 1600 mg of chloride.

In some embodiments, the formulation includes sufficient vitamins, minerals, essential fatty acids, absorbable carbohydrate, and absorbable nitrogen to meet metabolic demand as determined by the United States Department of Agriculture where the nutritional product may be used in metabolically stressed subjects expressing the symptomology of gastrointestinal inflammation diseases, as a sole source of nutrition.

### Pharmaceutical Compositions

In one aspect, the invention relates to compositions comprising the disclosed compounds and products of disclosed methods. For example, without limitation, a pharmaceutical composition may be provided comprising an effective amount of at least one disclosed compound, at least one product of a disclosed method, or a pharmaceutically acceptable salt, solvate, hydrate, or polymorph thereof, and a pharmaceutically acceptable carrier. In one aspect, the invention relates to pharmaceutical compositions comprising a pharmaceutically acceptable carrier and an effective amount of at least one disclosed compound; or a pharmaceutically acceptable salt, hydrate, solvate, or polymorph thereof.

In a further aspect, the effective amount is a therapeutically effective amount. In a still further aspect, the effective amount is a prophylactically effective amount. In a still further aspect, the pharmaceutical composition comprises a compound that is a product of a disclosed method of making.

In a further aspect, the pharmaceutical composition comprises a disclosed compound. In a yet further aspect, the pharmaceutical composition comprises a product of a disclosed method of making.

In one aspect, the pharmaceutical composition is used to treat a vertebrate. In a yet further aspect, the vertebrate is a human. In a further aspect, the vertebrate has been diagnosed with a need for treatment of the disorder prior to the administering step. In a further aspect, the vertebrate has been identified to be in need of treatment of the disorder.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and enteral (including introduction by any route into the gastrointestinal tract, voluntarily or mechanically, and in an appropriate form for the method of introduction/administration) administration, although the most suitable route in any given case will depend on the particular host, and nature and severity of the conditions for which the active ingredient is being administered. Pharmaceutical compositions may be conveniently presented in unit dosage form and prepared by any of the methods well known in the art of pharmacy.

### 1. Pharmaceutically Acceptable Salts

As used herein, the term “pharmaceutically acceptable salts” may refer to salts prepared from pharmaceutically acceptable non-toxic bases or acids. When the compound of the present invention is acidic, its corresponding salt may be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc, and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts may be formed include ion exchange resins such as, for example, arginine, betaine, caffeine, choline, N,N′-dibenzylethylenediamine, diethylamine, 2-di ethylaminoethanol, 2-dimethylaminoethanol, ethanolamine, ethylenediamine, N-ethylmorpho line, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids,” may include inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention may be combined as the active ingredient in intimate admixture with a pharmaceutical carrier according to conventional pharmaceutical compounding techniques. The carrier may take a wide variety of forms depending on the form of preparation desired for administration, e.g., oral, or parenteral (including intravenous). Thus, the pharmaceutical compositions of the present invention may be presented as discrete units suitable for oral administration such as capsules, cachets, or tablets each containing a predetermined amount of the active ingredient. Further, the compositions may be presented as a powder, as lyophilized powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, may also be administered by controlled release delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product may then be conveniently shaped into the desired presentation.

### 2. Pharmaceutical Carriers

Thus, the pharmaceutical compositions of this invention may include a pharmaceutically acceptable carrier and either a compound of the invention described herein or a pharmaceutically acceptable salt of the compounds of the invention. The compounds of the invention, or pharmaceutically acceptable salts thereof, may also be included in pharmaceutical compositions in combination with one or more other therapeutically active compounds.

The pharmaceutical carrier employed may be, for example, a solid, liquid, or gas. Examples of solid carriers may include lactose, terra alba, sucrose, talc, gelatin, agar, pectin, acacia, magnesium stearate, and stearic acid. Examples of liquid carriers may include sugar syrup, peanut oil, olive oil, and water. Examples of gaseous carriers may include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, and topical, and parenteral (including subcutaneous, intramuscular, and intravenous) administration, although the most suitable route in any given case will depend on the particular host and nature and severity of the conditions for which the active ingredient is being administered. Pharmaceutical compositions may be conveniently presented in unit dosage form and prepared by any of the methods well known in the art of pharmacy.

Pharmaceutical compositions of the present invention suitable for parenteral administration may be prepared as solutions or suspensions of the active compounds in water. A suitable surfactant may be included such as, for example, hydroxypropylcellulose. Dispersions may also be prepared in glycerol, liquid polyethylene glycols, and mixtures thereof in oils. Further, a preservative may be included to prevent the detrimental growth of microorganisms.

Pharmaceutical compositions of the present invention suitable for injectable use include sterile aqueous solutions or dispersions. Furthermore, the compositions may be in the form of sterile powders for the extemporaneous preparation of such sterile injectable solutions or dispersions. In all cases, the final injectable form must be sterile and must be effectively fluid for easy syringe-ability. The pharmaceutical compositions must be stable under the conditions of manufacture and storage; thus, preferably should be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), vegetable oils, and suitable mixtures thereof.

Pharmaceutical compositions of this invention may be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

### 3. Additional Ingredients

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants may be included to render the formulation isotonic with the blood of the intended recipient. Compositions containing a compound of the invention, and/or pharmaceutically acceptable salts thereof, may also be prepared in powder or liquid concentrate form.

### Example Formulation

Described below is an exemplary embodiment of a formulation in accordance with the present invention. The formulation is a nutritional product effective for treating IBD, such as, but not limited to, ulcerative colitis and Crohn's disease. In preferred embodiments, the nutritional product is liquid and orally ingestible.

Referring to Fig. #, an exemplary formulation according to the present invention includes the following ingredients: Water, Corn Maltodextrin, Whey Protein Hydrolysate, Hydrolyzed Sodium Caseinate, Mannan (forms), Medium Chain Triglycerides. Less than 1% of: Marine Oil (May Contain One or More of the Following: Anchovy, Cod, Menhaden, Pollock, Salmon, Sardine, Tuna), Sugar, Cellulose Gel, Magnesium Phosphate, Calcium Carbonate, Potassium Citrate, Sodium Phosphate, Canola Oil, Potassium Chloride, DATEM, Choline Chloride, Ascorbic Acid, Calcium Phosphate, Potassium Hydroxide, Carrageenan, Cellulose Gum, Magnesium Chloride, L-Carnitine, Taurine, Zinc Sulfate, Ferrous Sulfate, d-Alpha-Tocopheryl Acetate, Manganese Sulfate, Niacinamide, Calcium Pantothenate, Copper Sulfate, Thiamine Hydrochloride, Pyridoxine Hydrochloride, Riboflavin, Vitamin A Palmitate, Chromium Chloride, Folic Acid, Sodium Molybdate, Phylloquinone, Potassium Iodide, Sodium Selenate, Biotin, Vitamin D3, and Vitamin B12.

Provided in the table below are ingredient values per 8 fluid ounces (237 ml container) of the nutritional product, Q.S. to volume (with water). This formulation was 29.6 cal per ounce or 1 cal per mL.

The ingredients do not have to be combined in any particular order. However, order of addition may be based on bulkiness or on solubilization.

Ingredient Values per 8(eight) Fluid Ounces or 237 mL Container
- QS to Volume; 29.6 calories per ounce; 1 calorie per mL.

| INGREDIENT | VALUE |
|---|---|
| Calorie Additives | |
| PROTEIN, gm (as peptides and whole protein sources) | 21.0 |
| FAT, gm (as combined n3, n6, and other sources) | 6.0 |
| CARBOHYDRATE, gm | 24.5 |
| Other Carbohydrates Substances | |
| FIBER AS MANNAN AND OR MANNAN DERIVATIVE, gm | 5.3 |
| TOTAL SUGARS, gm | 3.0 |
| Amino Acids | |
| L-CARNITINE, mg | 22.0 |
| TAURINE, mg | 26.0 |
| GLUTAMINE, g | 4.0 |
| CYSTINE, mg | 400 |
| Vitamins and Vital Compounds | |
| VITAMINE A, mcg (as RAE value) | 170.0 |
| VITAMIN D, mcg | 6.0 |
| VITAMIN E, mg (as D-alpha tocopherol) | 5.0 |
| VITAMIN K, mcg (as VITAMIN K2) | 32.0 |
| VITAMIN C, mg | 100.0 |
| FOLATE, mcg (as DFE + Folic Acid values) | 170.0 |
| THIAMINE, mg (Vitamin B1) | 0.60 |
| RIBOFLAVIN, mg (Vitamin B2) | 0.35 |
| PYRIDOXINE, mg (Vitamin B6) | 0.50 |
| NIACIN, mg (Vitamin B3) | 7.0 |
| CYANOCOBALAMIN, mcg (Vitamin B12) | 50.0 |
| CHOLINE, mg | 120.0 |
| BIOTIN, mcg | 10.0 |
| PANTOTHENIC ACID, mg | 1.5 |
| Salts, Minerals, Trace Elements | |
| SODIUM, mg | 325.0 |
| POTASSIUM, mg | 327.0 |
| CHLORIDE, mg | 243.0 |
| CALCIUM, mg | 210.0 |
| PHOSPHORUS, mg | 202.0 |
| MAGNESIUM, mg | 65.0 |
| IODINE, mg | 25.0 |
| MANGANESE, mg | 0.75 |
| COPPER, mg | 0.40 |
| ZINC, mg | 4.5 |
| IRON, mg | 2.9 |
| SELENIUM, mcg | 16.0 |
| CHROMIUM, mcg | 18.0 |
| MOLYBDENUM, mcg | 30.0 |
| COBALT (as part of Vitamin B12), mcg | 2.0 |
| BORON, mg | 1.5 |

g or gm = gram wt.; mg = milligram wt.; mcg = microgram wt.
Flavoring may be added as requested for product variance.

In other embodiments of the nutritional product, adjustments can be made to achieve different caloric densities within the same volume (8 fluid ounces). For example, an embodiment of the nutritional product may include 28.0 g of protein, 9.0 g of fat, and 32 g of digestible carbohydrate, Q. S. to volume; this formulation would have about 1.3 calories per milliliter. In another example, an embodiment of the nutritional product may include 30.0 g of protein, 10.5 g of fat, and 35 g of digestible carbohydrate, Q. S. to volume; this formulation would have about 1.5 calories per milliliter.

Preferably, the pH of the formulation is in the range of 6.20 to 6.85.

In embodiments of the invention, the caloric density may vary depending on the level of usage of the nutritional product and/or the use of the product as a dietary replacement. Preferably, the caloric density of the formulation ranges from about 1.0 to about 1.5 calories per ml. Caloric density can be varied based on concentration of the or additional hydrolyzed protein and/or the addition of short-chain fatty acids and/or monounsaturated fat. This is intended to provide more nutrients in a smaller volume for person who cannot consume larger volumes and who wish to consume other foods where the drink is a meal supplement and not a replacement.

The specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

It is recommended that this product be administered at a level to achieve mitigation of inflammatory bowel disease markers such as, but not limited to, C-reactive protein, stool frequency, C-protectin (calprotectin), or other clinical marker of disease or quality of life associated with the disease.

In this embodiment, the dry volume of the mannan or mannan derivatives can be determined based on the dry weight of the final ingredient list (dry ingredients in toto), which excludes water, oils, and oil-based ingredients (e.g., vitamin D, E, and A). It includes the protein (21 g), fat (6 g), carbohydrate (24.5 g), fiber (5.3 g) sugars (3.0 g), and glutamine (4 g).

In this embodiment, the ratio of marine oil to canola oil was approximately 2:1.

In this embodiment, the relative amounts of, carbohydrates, non-essential amino acids, vitamins/vital compounds, and salts/minerals/trace elements are set so that the formulation can meet the level of nutrients as a daily sole source of nutrition when consumed as 5 units a day. This may depend on container volume, but each ounce will be exactly the same regardless of container volume.

Imaging and Diagnosis 1. Optical Imaging

The disclosed compounds may include a detectable label useful for optical imaging. A number of approaches may be used for optical imaging. The various methods may depend upon fluorescence, bioluminescence, absorption, or reflectance as the source of contrast. Fluorophores are compounds or moieties that absorb energy of a specific wavelength and re-emit energy at a different (but equally specific) wavelength. In certain embodiments, the detectable label is a near-infrared (NIR) fluorophore. Suitable NIRs include, but are not limited to, VivoTag-S® 680 and 750, Kodak X-SIGHT Dyes and Conjugates, DyLight 750 and 800 Fluors, Cy 5, Cy 5.5 and 7 Fluors, Alexa Fluor 680 and 750 Dyes, Alexa Fluor 688, and IRDye 680 and 800CW Fluors and combinations thereof. In certain embodiments, Quantum dots, with their photostability and bright emissions, may also be used with optical imaging.

### 2. Nuclear Medicine Imaging

The disclosed compounds may include a detectable label (e.g., a radionuclide) useful for nuclear medicine imaging. Nuclear medicine imaging involves the use and detection of radioisotopes in the body. Nuclear medicine imaging techniques include scintigraphy, single photon emission computed tomography (SPECT), and positron emission tomography (PET). In these techniques, radiation from the radioisotopes may be captured by a gamma camera to form two-dimensional images (scintigraphy) or 3-dimensional images (SPECT and PET).

Radioisotopes that may be incorporated into or attached directly to the disclosed compounds include, but are not limited to, tritium, ¹¹C, ¹³N, ¹⁴C, ¹⁵O, ¹⁸Fl, ⁶²Cu, ⁶⁴Cu, ⁶⁷Cu, ⁶⁸Ga, ⁷⁶Br, ⁸²Rb, ⁹⁰Y, ^{99m}Tc, ¹¹¹In, ¹²³I, ¹²⁴I, ¹²⁵I, ¹³¹I, ¹⁵³Sm, ²⁰¹Tl, ¹⁸⁶Re, ¹⁸⁸Re, ^{117m}Sn and ²¹²Bi. In certain embodiments, the radioisotope is attached to a disclosed compound by halogenation. Radionuclides used in PET scanning are typically isotopes with short half-lives. Typical isotopes include ¹¹C, ¹³N, ¹⁵O, ¹⁸F, ⁶⁴Cu, ⁶²Cu, ¹²⁴I, ⁷⁶Br, ⁸²Rb and ⁶⁸Ga, with ¹⁸F being the most clinically utilized.

Gamma radiation from radioisotopes may be detected using a gamma particle detection device. In some embodiments, the gamma particle detection device is a Gamma Finder® device (SenoRx, Irvine Calif.). In some embodiments, the gamma particle detection device is a Neoprobe® GDS gamma detection system (Cincinnati, Ohio).

Positron emission tomography is a nuclear medicine imaging technique which produces a three-dimensional image or picture of functional processes in the body. Some agents used for PET imaging provide information about tissue metabolism or some other specific molecular activity. Commonly used agents or potential agents that may be used as detectable agents include but are not limited to: ⁶⁴Cu diacetyl-bis(N⁴-methylthiosemicarbazone); ¹⁸F-fluorodeoxyglucose (FDG); ¹⁸F-fluoride; 3′-deoxy-3′-[¹⁸F]fluorothymidine (FLT); ¹⁸F-fluoromisonidazole; Gallium; Technetium-99m; and Thallium. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerythrin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

A number of trivalent metal radionuclides have physical properties suitable for radioisotope imaging (e.g., indium-111 (¹¹¹In) gallium-67/68 (67/68Ga) and yttrium-86 (⁸⁶Y)) or for targeted radionuclide therapy (e.g., ⁹⁰Y and lutetium-177 (¹⁷⁷Lu)). Diethylenetriaminepentaacetic acid (DTPA) and/or 1,4,7,10-tetraazacyclododecane-1,4,7,10-tetraacetic acid (DOTA; CAS 60239-18-1) may be used.

### 3. Magnetic Resonance Imaging

The disclosed compounds may be detected via magnetic resonance imaging. MRI has the advantages of having remarkably high spatial resolution and is very adept at morphological imaging and functional imaging. MRI generally has a sensitivity of around one milli-mol/L to 10 micro-mol/L. Improvements to increase MR sensitivity include hyperpolarization by increasing magnetic field strength, optical pumping, or dynamic nuclear polarization. There are also a variety of signal amplification schemes based on chemical exchange that increase sensitivity.

### 4. Actions Based on Imaging and Identifications

The disclosed methods include the determination, identification, indication, correlation, diagnosis, prognosis, etc. (which may be referred to collectively as “identifications”) of subjects, diseases, conditions, states, etc. based on imaging, measurements, detections, comparisons, analyses, assays, screenings, etc. For example, the disclosed imaging methods may enable the identification of patients, organs, tissues, etc. having cancer cells, metastasized cancer cells, cancer cells beyond tumor margins, etc. With this knowledge, it may be possible to determine specific actions (e.g., treatment, behaviors, etc.) that may be taken to address the cancer (i.e., cancer cells, metastasized cancer cells, cancer cells beyond tumor margins) that may be suitable for the identified patents, organs, tissues, etc., but not for others (e.g., subjects not diagnosed with a particular disease or condition). This ability to differentiate between who would benefit from the specific actions, and who would not, may thereby facilitate the efficient administration of treatment by ensuring that only those who need or would otherwise benefit from out will receive it, and those who do not need or would otherwise benefit from it will not receive it. Of course, this is just one non-limiting example as such identifications are useful for many other reasons.

Accordingly, also disclosed herein are methods that comprise taking particular actions following and based on the disclosed identifications. For example, disclosed are methods that comprise creating a record of an identification in a physical (e.g., paper), electronic, or other form. Thus, creating a record of an identification based on the disclosed methods differs physically and tangibly from merely performing an imaging, measurement, detection, comparison, analysis, assay, screen, etc. Such a record is particularly substantial and significant in that it allows the identification to be fixed in a tangible form that may be, for example, communicated to others (such as those who could treat, monitor, follow-up, advise, etc. the subject based on the identification); retained for later use or review; used as data to assess sets of subjects, treatment efficacy, accuracy of identifications based on different imaging, measurements, detections, comparisons, analyses, assays, screenings, etc., and the like. Such uses of records of identifications may be made, for example, by the same individual or entity as, by a different individual or entity than, or a combination of the same individual or entity as and a different individual or entity than, the individual or entity that made the record of the identification. The disclosed methods of creating a record may be combined with any one or more other methods disclosed herein, and in particular, with any one or more steps of the disclosed methods of identification.

As another example, disclosed are methods that comprise making one or more further identifications based on one or more other identifications. For example, particular treatments, monitoring, follow-up, advice, etc. may be identified based on the other identification. For example, identification of a subject that has a disease or condition with a high level of a particular component or characteristic may be further identified as a subject that could or should be treated with a therapy based on or directed to the high-level component or characteristic. A record of such further identifications may be created (as described above, for example) and may be used in any suitable way. Such further identifications may be based, for example, directly on the other identifications, a record of such other identifications, or a combination. Such further identifications may be made, for example, by the same individual or entity as, by a different individual or entity than, or a combination of the same individual or entity as and a different individual or entity than, the individual or entity that made the other identifications. The disclosed methods of making a further identification may be combined with any one or other methods disclosed herein, and in particular, with any one or more steps of the disclosed methods of identification.

As another example, disclosed are methods that comprise treating, monitoring, following-up with, advising, etc. a subject identified in any of the disclosed methods. Also disclosed are methods that comprise treating, monitoring, following-up with, advising, etc. a subject for which a record of an identification from any of the disclosed methods has been made. For example, particular treatments, monitoring, follow-up, advice, etc. may be used based on an identification and/or based on a record of an identification. For example, a subject identified as having a disease or condition with a high level of a particular component or characteristic (and/or a subject for which a record has been made of such an identification) may be treated with a therapy based on or directed to the high-level component or characteristic. Such treatments, monitoring, follow-ups, advice, etc. may be based, for example, directly on identifications, a record of such identifications, or a combination. Such treatments, monitoring, follow-ups, advice, etc. may be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. may be combined with any one or more other methods disclosed herein, and in particular, with any one or more steps of the disclosed methods of identification.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A liquid nutritional product for enteral delivery of a C-type lectin receptor ligand intended for the mitigation of inflammatory gastrointestinal diseases involving C-type lectin receptor-expressing inflammatory cells where such nutritional product is comprised of:
A] a first source of protein including a soy protein hydrolysate having a molecular weight partition as determined by appropriate sizing procedures, where in not less than 65% of the particles have a molecular weight in the range of 700 to 7000 Da;
B] a second source of protein which comprises a source of intact protein in a quantity sufficient to yield a stable emulsion and where in its inclusion fulfills a satisfactory and metabolically appropriate amino acid profile;
C] a compatible emulsifying agent selected among those of soy lecithin, egg yolk lecithin, or sunflower lecithin;
D] a source of fat wherein the sum of the n-3 fatty acids to n-6 fatty acids approximates a 30-45% of n-3 to this mixture;
E] a source of mannan or mannan derivatives wherein mannan or the explicit mannan derivatives whether alone or in combination have a molecular weight distribution of 100 Da to 200,000 Da and where in mannan and or derivatives comprise 1% and 20% of the dry weight of the final ingredient list (dry ingredients in toto).

2. A nutritional product for the support of persons expressing the symptomology of gastrointestinal inflammation diseases described in claim 1 where the source of fat for this formulation includes fish oil.

3. A nutritional product for the support of persons expressing the symptomology of gastrointestinal inflammation diseases described in claim 2 where the source of protein comprises sodium caseinate.

4. A nutritional product for the support of persons expressing the symptomology of gastrointestinal inflammation diseases as described in claim 1 further comprising a suitable mixture of mannan and mannan derivatives wherein mannan and mannan derivatives comprise 2 to 10% of the dry weight of the final ingredient list ((dry ingredients; referring to ingredients absent any solvent or dissolution ingredient).

5. A nutritional product for the support of persons expressing the symptomology of gastrointestinal inflammation diseases described in claim 4 where the source of protein comprises sodium caseinate.

6. A nutritional product for the support of persons expressing the symptomology of gastrointestinal inflammation diseases as described in claim 4 further comprising a suitable mixture of mannan and mannan derivatives wherein mannan and mannan derivatives comprise 4 to 8% of the dry weight of the final ingredient list (dry ingredients; referring to ingredients absent any solvent or dissolution ingredient).

7. A nutritional product for the support of persons expressing the symptomology of gastrointestinal inflammation diseases described in claim 6 where the source of protein comprises sodium caseinate.

8. A nutritional product for the support of persons expressing the symptomology of gastrointestinal inflammation diseases as described in claim 1 wherein the soy protein hydrolysate has a degree of hydrolysis in the range of 12 to 20 and the amino acid profile of the soy protein hydrolysate is less than 3% free amino acids.

9. A nutritional product for the support of persons expressing the symptomology of gastrointestinal inflammation diseases as described in claim 8 wherein the intact protein comprises sodium caseinate.

10. A nutritional product for the support of persons expressing the symptomology of gastrointestinal inflammation diseases as described in claim one wherein the intact protein is selected from the group consisting of sodium caseinate, whey protein isolate, fish protein isolate, potato protein isolate, and or avian egg albumin isolate.

11. A nutritional product for the support of persons expressing the symptomology of gastrointestinal inflammation diseases as described in claim 1 wherein the nutritional product is a caloric density of about 1.0 to 1.5 calories per ml.

12. A nutritional product for the support of persons expressing the symptomology of gastrointestinal inflammation diseases:
a) a first source of protein comprising a soy protein hydrolysate having a degree of hydrolysis in the range of 12 to 20 and a molecular weight partition, as it determined by suitable qualitative analyses, wherein 25% to 65% of the oligopeptides and polypeptides have a molecular weight range of 1200 to 7500 Da in the amino acid profile of the soy protein hydrolysate has less than 2% free amino acids;
b) a second source of protein which comprises sodium caseinate, the sodium caseinate comprising by weight of approximately 8 to 28% of the protein in the nutritional product;
c) a source of emulsifying agents that include but are not limited to soy lecithin, egg yolk lecithin, and or sunflower lecithin;
d) a source of fat characterized by the ratio (w/w) of the total n-6 fatty acids to the sum of the n-3 fatty acids being in the range of about 1.5:1 to 3:1; the nutritional product having a pH in the range of 6.20 to 6.85 and being a stable emulsion after being subjected to production processing.

13. A nutritional product for the support of persons expressing the symptomology of gastrointestinal inflammation diseases as described in claim 12 further comprising a suitable mixture of mannan and mannan derivatives wherein mannan and mannan derivatives comprise 2 to 10% of the dry weight of the final ingredient list ((dry ingredients; referring to ingredients absent any solvent or dissolution ingredient).

14. A nutritional product for the support of persons expressing the symptomology of gastrointestinal inflammation diseases as described in claim 12 where a source of fat comprises fish oil.

15. A nutritional product for the support of persons expressing the symptomology of gastrointestinal inflammation diseases as described in claim 12 wherein the source of fat comprises fish oil and or canola oil and/or avocado oil and medium chain triglycerides.

16. A nutritional product for the support of persons expressing the symptomology of gastrointestinal inflammation diseases as described in claim 12 wherein the nutritional product is a caloric density of about 1.0 to 1.50 calories per ml.

17. A nutritional product for the support of persons expressing the symptomology of gastrointestinal inflammation diseases:
a) a first source of protein comprising a soy protein hydrolysate having a degree of hydrolysis in the range of 12 to 20 and a molecular weight partition, as it determined by suitable qualitative analyses, wherein 25% to 65% of the oligopeptides and polypeptides have a molecular weight range of 1200 to 7500 Da in the amino acid profile of the soy protein hydrolysate has less than 2% free amino acids;
b) a second source of protein which comprises sodium caseinate, the sodium caseinate comprising by weight of approximately 8 to 28% of the protein in the nutritional product;
c) a source of emulsifying agents that include but are not limited to soy lecithin, egg yolk lecithin, and or sunflower lecithin;
d) a source of fat selected from and including fish oil and or canola oil and/or avocado oil and medium chain triglycerides characterized by the ratio (w/w) of the total n-6 fatty acids to the sum of the n-3 fatty acids being in the range of about 1.5:1 to 3:1;
e) A nutritional product for the support of persons expressing the symptomology of gastrointestinal inflammation diseases comprising a suitable mixture of mannan and mannan derivatives wherein mannan and mannan derivatives comprise 2 to 10% of the dry weight of the final ingredient list ((dry ingredients; referring to ingredients absent any solvent or dissolution ingredient), the nutritional product having a caloric density in the range of 1.0 to 1.50 calories per ml; the nutritional product having a pH in the range of 6.20 to 6.85 and being a stable emulsion after being subjected to production processing.

18. A nutritional product for the support of persons expressing the symptomology of gastrointestinal inflammation diseases as described in claim 17, further comprising selected carotenoids, anthocyanins and antioxidants.

19. A nutritional product for the support of persons expressing the symptomology of gastrointestinal inflammation diseases as described in claim 17 further comprising (on a per liter basis) and approximate isotonicity balance: (a) 950 to 1100 mg of sodium; (b) 2400 to 2650 mg of potassium; and (c) about 1250 to 1600 mg of chloride.

20. A nutritional product for the support of persons expressing the symptomology of gastrointestinal inflammation diseases as described in claim 17 further comprising sufficient vitamins, minerals, essential fatty acids, absorbable carbohydrate, and absorbable nitrogen to meet metabolic demand as determined by the United States Department of Agriculture where the nutritional product may be used in metabolically stressed subjects expressing the symptomology of gastrointestinal inflammation diseases, as a sole source of nutrition.
